(19) United States
(12) Patent Application Publication
(10) Pub. No.: US 2020/0088820 A1
(43) Pub. Date: Mar. 19, 2020

(54) PHASE OFFSET ERROR CORRECTION FOR DISPLACEMENT-ENCODED MR IMAGES

(71) Applicants: Siemens Healthcare GmbH, Erlangen (DE); Emory University, Atlanta, GA (US)

(72) Inventors: Xiaodong Zhong, Lilburn, GA (US); John Oshinski, Decatur, GA (US); Ning Jin, Powell, OH (US)

(21) Appl. No.: 16/132,574

(22) Filed: Sep. 17, 2018

(51) Int. Cl. G01R 33/46; G01R 33/48; G01R 33/563; G01R 33/565; A61B 5/055

(52) U.S. Cl. CPC G01R 33/4625; G01R 33/4818; A61B 5/055; G01R 33/56509; G01R 33/56316

(57) ABSTRACT

A system includes acquisition of a plurality of displacement-encoded magnetic resonance (MR) phase images, determination of first pixels associated with one or more image regions of the plurality of displacement-encoded MR phase images, determination of representative pixel values of the first pixels based on pixel values of the first pixels within one or more of the plurality of displacement-encoded MR phase images, determination of a relationship between background phase offset error and pixel location based on the determined representative pixel values and the pixel locations of the first pixels, determination of a background phase offset error for each of one or more other pixels of the plurality of displacement-encoded MR phase images based on the relationship and on the pixel locations of the one or more other pixels, and generation of a corrected MR phase image of one of the plurality of displacement-encoded MR phase images based on the background phase offset error determined for each of the one or more other pixels.

### BACKGROUND

The presence of disease may change one or more mechanical characteristics of tissue. Quantitative imaging may be used to determine such characteristics and to thereby assist in the diagnosis and evaluation of disease. Systems for accurately determining mechanical characteristics via imaging are therefore desired.

Displacement Encoding with Stimulated Echoes (DENSE) is a magnetic resonance (MR) imaging technique for quantitative imaging of moving tissue. More specifically, the DENSE technique encodes tissue displacement into acquired MR phase images. Displacement or motion values at each pixel location are extracted from the MR phase images for each displacement-encoded direction, and these values can be combined to generate a tissue displacement map as shown in FIG. 1. The displacement map can be used to calculate mechanical characteristics/indices of the tissue.

Background phase offset errors in the phase images can compromise the accuracy of the determined displacement values. These errors may be caused by, for example, system-imperfect factors such as spatial nonlinearity of gradients and eddy current. These errors may be significant in the case of small displacements such as brain motion, which would substantially affect the accuracy and usefulness of the extracted displacement values and any subsequent index calculations.

Conventional systems generate background phase offset error maps using a stationary phantom. The stationary phantom is imaged using a same DENSE sequence and clinical parameters as those used to image a patient volume. The DENSE phase images of the phantom-based acquisition are subtracted from the DENSE phase images of a patient scan to obtain corrected phase images of the patient volume. This technique requires extra clinical effort and resources, and is subject to errors due to differences between the phantom and the patient volume, and any other differences which may affect the background phase offset error associated with each scan.

### BRIEF DESCRIPTION OF THE DRAWINGS

FIG. 1 illustrates a displacement map generated based on displacement-encoded MR images.

FIG. 2 is a block diagram of a system to determine displacement based on displacement-encoded MR images according to some embodiments.

FIG. 3 comprises a flow diagram of a process to determine background phase offset error according to some embodiments.

FIG. 4 is a block diagram of a system to generate magnitude and phase images based on MR data according to some embodiments.

FIG. 5 illustrates pulse sequences for acquiring displacement-encoded MR images according to some embodiments.

FIG. 6 illustrates generation of corrected phase images based on determined pixel-specific background phase offset errors according to some embodiments.

FIG. 7 is a block diagram of an MR system according to some embodiments.

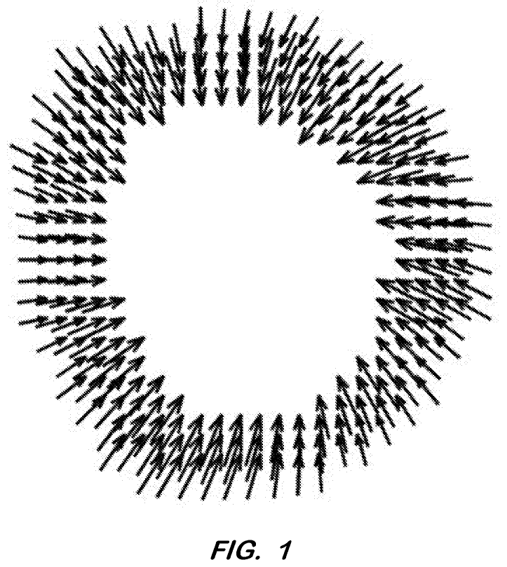

### DETAILED DESCRIPTION

The following description is provided to enable any person in the art to make and use the described embodiments. Various modifications, however, will remain readily apparent to those in the art.

Generally, some embodiments provide background phase error correction of MR displacement-encoded phase images without the acquisition of separate phantom images. Some embodiments estimate a spatially-varying background phase error from acquired MR images and correct the phase images using the estimated background phase error. As will be described below, the estimation may exploit the relatively slow variation of background phase error over space and time. The displacement values extracted from the corrected phase images, and any indices or diagnosis determined therefrom, may be more accurate than those extracted or determined using the original non-corrected phase images.

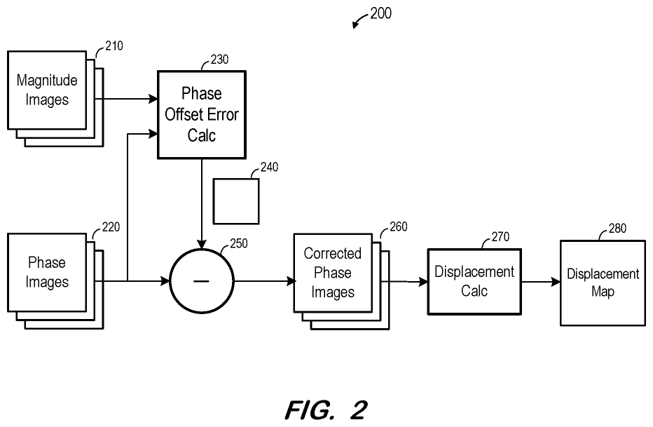

FIG. 2 is a block diagram of a system to determine displacement based on displacement-encoded MR images and on background phase error correction according to some embodiments.

System 200 shows magnitude images 210 and corresponding displacement-encoded phase images 220. Each magnitude image 210 and corresponding phase image 220 is associated with a respective time period, such that magnitude images 210 and corresponding displacement-encoded phase images 220 comprise time-series data. Magnitude images 210 and corresponding displacement-encoded phase images 220 are generated based on k-space data acquired during MR imaging as is known in the art. The acquisition of the k-space data is performed using known displacement-encoding acquisition techniques (e.g., DENSE) such that phase images 220 encode tissue displacement which may have occurred during image acquisition. Acquisition of the k-space data and generation of magnitude images 210 and corresponding displacement-encoded phase images 220 based on the k-space data will be described in more detail below.

Phase offset error calculation unit 230 receives magnitude images 210 and corresponding displacement-encoded phase images 220 and determines a background phase offset error. Briefly, according to one example, image pixel locations corresponding to noise regions are determined by applying a threshold to magnitude images 210. Pixel locations corresponding to stationary regions are identified based on pixels of phase images 220 which exhibit the least phase change over time.

Phase offset error calculation unit 230 then generates a mathematical representation which fits the pixel values of the stationary pixels as a function of pixel location, and may determine a phase offset error 240 for each image pixel location (or at least for each pixel location in a non-stationary image region) based on the mathematical representation. Phase offset error 240 is subtracted 250 from each of phase images 220 to generate corrected phase images 260. Next, displacement calculation unit 270 generates displacement map 280 based on corrected phase images 260.

Each functional unit described herein may be implemented at least in part in computer hardware, in program code and/or in one or more computing systems including executing such program code as is known in the art. Such a computing system may include one or more processing units which execute processor-executable program code stored in a memory system. A single computing system may implement two or more functional units described herein.

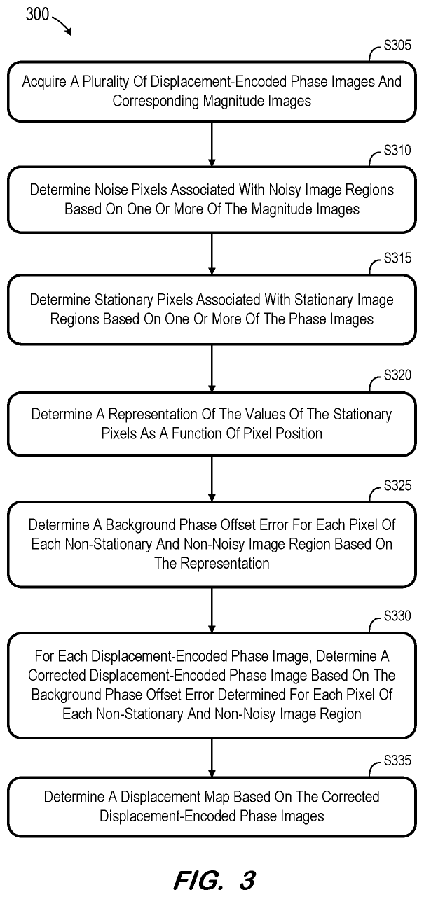

FIG. 3 comprises a flowchart of process 300 according to some embodiments. In some embodiments, various hardware elements of one or more computing systems execute program code to perform process 300. Process 300 and all other processes mentioned herein may be embodied in processor-executable program code read from one or more of non-transitory computer-readable media, such as a floppy disk, a disk-based or solid-state hard drive, CD-ROM, a DVD-ROM, a Flash drive, and a magnetic tape, and then stored in a compressed, uncompiled and/or encrypted format. In some embodiments, hard-wired circuitry may be used in place of, or in combination with, program code for implementation of processes according to some embodiments. Embodiments are therefore not limited to any specific combination of hardware and software.

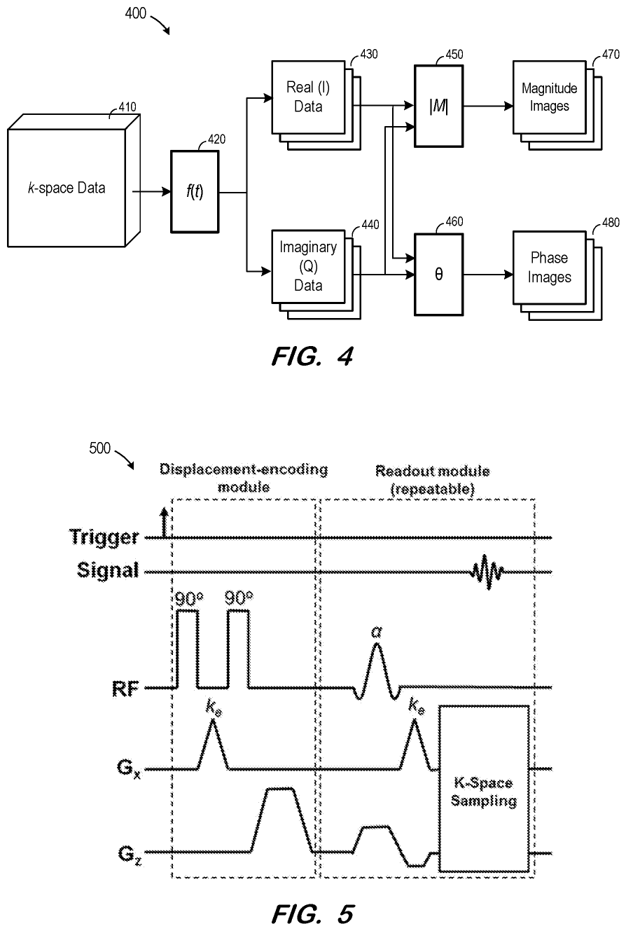

Initially, at S305, a plurality of displacement-encoded phase images and corresponding magnitude images are acquired. According to some embodiments, S305 includes execution of an MR imaging scan to acquire k-space data and generation of the plurality of displacement-encoded phase images and corresponding magnitude images based on the k-space data. FIG. 4 illustrates generation of displacement-encoded phase images and corresponding magnitude images according to some embodiments.

As described above, S305 may include acquisition of k-space data 410 using an MR imaging system. k-space data 410 consists of complex values in spatial frequency domain which are sampled based on a predefined sequence of radiofrequency and gradient pulses. It is assumed that k-space data 410 of FIG. 4 are acquired using a displacement encoding acquisition scheme which encodes positional information into the phase of each image pixel.

The DENSE acquisition technique, to which embodiments are not limited, provides high spatial density of displacement measurements via stimulated echoes. The technique encodes motion over long time intervals. To encode displacement over a time comparable to T₁ while avoiding T*₂-related signal decay, stimulated echoes are used to store the magnetization vector along the direction of the static magnetic field.

FIG. 5 illustrates pulse sequence 400 according to DENSE. After an initial RF excitation, phase dispersion is introduced using a single gradient lobe along the desired direction. For example, along the read direction Gₓ the gradient pulse was set at a displacement encoding frequency of kₑ in the unit of cycles/mm which is proportional to the gradient area. Subsequently, a second RF pulse is applied to preserve the magnetization along the longitudinal axis. Displacement as encoded during a long mixing period followed by a third RF pulse to bring the magnetization onto the transverse plane. Then, a second gradient pulse of the same displacement encoding frequency of kₑ rewinds the phase dispersion from the first lobe. For stationary spins this phase rewinding is complete. For spins that move Δx during this period, a phase of φ₁=(2πkₑΔx+φ₀) is accumulated, where φ₀ is the phase value when the encoding gradient is set to 0 and NOT the background phase offset error measured later. Imaging is performed with slice selection during the third RF pulse followed by sequential k-space sampling. The sequence is repeated once more with the displacement-encoding gradient pulses set to 0. The accumulated phase is then φ₀. The phase difference between the images being Δφ₁=φ₁−φ₀=2πkₑΔx.

The equation Δφ₁=φ₁−φ₀=2πkₑΔx is used to measure Δx, since other phase contributions common to both images are canceled. To measure displacement along the read and slice directions, the corresponding encoding gradient amplitudes are modified accordingly.

Each complex data point of k-space data 410 includes a “real” part (I) and an “imaginary” part (Q). Inverse Fourier transform unit 420 converts k-space data 410 into real (I) data 430 and imaginary (Q) data 440 as is known in the art. Next, magnitude images 470 and phase images 480 are generated based on real (I) data 430 and imaginary (Q) data 440 as is also known in the art. For example, magnitude image calculation unit 450 may calculate magnitude images 470 as √(Real²+Imaginary²) for the complex data point at each image pixel, while phase image calculation unit 460 may calculate phase images 480 as tan⁻¹(Imaginary/Real) for the complex data point at each image pixel.

Returning to process 300, image pixels corresponding to noisy image regions are determined based on one or more of the acquired magnitude images. With reference to FIG. 4, and according to some embodiments, an average magnitude image is calculated by determining an average pixel value of magnitude images 470 for each image pixel. Assuming that noisy image regions of a magnitude image are dark, the pixels associated with average pixel values below a threshold are determined to be noise pixels at 310. In some embodiments, the determination at S310 includes manual identification of noise pixels by an operator.

At S315, stationary pixels associated with stationary image regions are determined based on the one or more acquired phase images. In some embodiments, the standard deviation in phase value of each image pixel over time is determined from phase images 480. Image pixels for which the standard deviation is below a threshold are determined to be stationary pixels at S315. Accordingly, “stationary” regions may not be completely stationary during the acquisition of the k-space data, but may be considered substantially stationary which, in some embodiments, is defined as having a standard deviation in pixel phase value which falls below a threshold.

A representation of the values of the stationary pixels as a function of pixel position is determined at S320. Since the stationary pixels are ideally associated with a zero value in the phase images, any values associated therewith are assumed to represent background phase offset error. Accordingly, the representation determined at S320 is a spatial representation of background phase offset error.

In some examples, a linear equation [e.g., Offset_{x,y}=Ax+By+C], a second-order equation [e.g., Offset_{x,y}=Ax²+By²+Cx+Dy+E], or any other type of equation may be used to represent the values of the stationary pixels as a function of pixel position. The coefficients of the equation may be solved using values of the stationary pixels of one of the phase images, values of the stationary pixels of an average phase image determined based on one or more of the phase images, or another set of pixel values determined based on the phase images.

A background phase offset error is determined for a plurality of image pixels at S325. A background phase offset error for a pixel may be determined by substituting the x-y position of the pixel into the solved representation determined at S320. The plurality of image pixels for which the background phase offset error is determined may comprise non-noise and non-stationary pixels (i.e., image pixels other than the pixels determined at S310 and S315). In some embodiments, the background phase offset error is determined for noise pixels and/or non-stationary pixels as well.

For each phase image, the determined background phase offset error of each pixel is subtracted from the value of the pixel in the phase image at S330. The subtraction may occur for all image pixels if the background phase offset error is determined for all pixels at S325. In some embodiments, the subtraction occurs only with respect to the non-noise and non-stationary pixels.

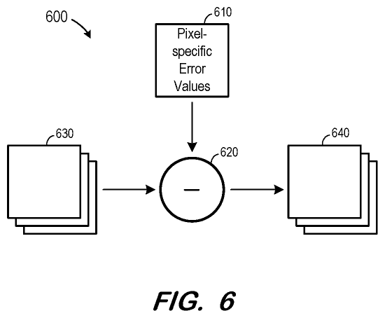

FIG. 6 illustrates S330 according to some embodiments. Pixel-specific error values 610 represent the background phase offset errors determined at S325. Subtraction unit 620 subtracts the values of values 610 from corresponding pixel values of each of phase images 630 to generated corrected phase images 640. A displacement map may be determined at S335 based on the resulting corrected phase images as is known in the art.

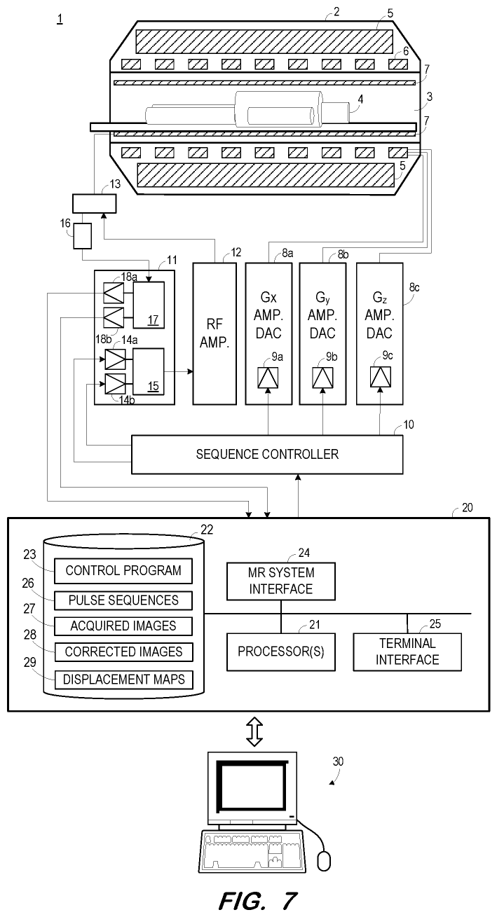

FIG. 7 illustrates MRI system 1 according to some embodiments. MRI system 1 includes MRI chassis 2, which defines bore 3 in which patient 4 is disposed. MRI chassis 2 includes polarizing main magnet 5, gradient coils 6 and RF coil 7 arranged about bore 3. According to some embodiments, polarizing main magnet 5 generates a uniform main magnetic field (B₀) and RF coil 7 emits an excitation field (B₁).

According to MRI techniques, a substance (e.g., human tissue) is subjected to a main polarizing magnetic field (i.e., B₀), causing the individual magnetic moments of the nuclear spins in the substance to process about the polarizing field in random order at their characteristic Larmor frequency, in an attempt to align with the field. A net magnetic moment M_{z} is produced in the direction of the polarizing field, and the randomly-oriented magnetic components in the perpendicular plane (the x-y plane) cancel out one another.

The substance is then subjected to an excitation field (i.e., B₁) created by emission of a radiofrequency (RF) pulse, which is in the x-y plane and near the Larmor frequency, causing the net aligned magnetic moment M_{z} to rotate into the x-y plane so as to produce a net transverse magnetic moment Mₜ, which is rotating, or spinning, in the x-y plane at the Larmor frequency. The excitation field is terminated and signals are emitted by the excited spins as they return to their pre-excitation field state. The emitted signals are detected, digitized and processed to reconstruct an image using one of many well-known MRI reconstruction techniques.

An RF pulse may be emitted as a magnetization preparation step in order to enhance or suppress signals from certain tissue so as to generate desired levels of contrast in the resulting image. For example, an inversion, or saturation, pulse is used in non-contrast-enhanced angiography to suppress venous blood in order to highlight the arterial system.

Gradient coils 6 produce magnetic field gradients Gₓ, G_{y}, and G_{z} which are used for position-encoding NMR signals. The magnetic field gradients Gₓ, G_{y}, and G_{z} distort the main magnetic field in a predictable way so that the Larmor frequency of nuclei within the main magnetic field varies as a function of position. Accordingly, an excitation field B₁ which is near a particular Larmor frequency will tip the net aligned moment M_{z} of those nuclei located at field positions which correspond to the particular Larmor frequency, and signals will be emitted only by those nuclei after the excitation field B₁ is terminated.

Gradient coils 6 may consist of three windings, for example, each of which is supplied with current by an amplifier 8a-8c in order to generate a linear gradient field in its respective Cartesian direction (i.e., x, y, or z). Each amplifier 8a-8c includes a digital-analog converter 9a-9c which is controlled by a sequence controller 10 to generate desired gradient pulses at proper times.

Sequence controller 10 also controls the generation of RF pulses by RF system 11 and RF power amplifier 12. RF system 11 and RF power amplifier 12 are responsive to a scan prescription and direction from sequence controller 10 to produce RF pulses of the desired frequency, phase, and pulse amplitude waveform. The generated RF pulses may be applied to the whole of RF coil 7 or to one or more local coils or coil arrays. RF coil 7 converts the RF pulses emitted by RF power amplifier 12, via multiplexer 13, into a magnetic alternating field in order to excite the nuclei and align the nuclear spins of the object to be examined or the region of the object to be examined. As mentioned above, RF pulses may be emitted in a magnetization preparation step in order to enhance or suppress certain signals.

The RF pulses are represented digitally as complex numbers. Sequence controller 10 supplies these numbers in real and imaginary parts to digital-analog converters 14a-14b in RF system 11 to create corresponding analog pulse sequences. Transmission channel 15 modulates the pulse sequences with a radio-frequency carrier signal having a base frequency corresponding to the resonance frequency of the nuclear spins in the volume to be imaged.

RF coil 7 both emits radio-frequency pulses as described above and scans the alternating field which is produced as a result of precessing nuclear spins, i.e. the nuclear spin echo signals. The received signals are received by multiplexer 13, amplified by RF amplifier 16 and demodulated in receiving channel 17 of RF system 11 in a phase-sensitive manner. Analog-digital converters 18a and 18b convert the demodulated signals into a real part and an imaginary part.

Computing system 20 receives the real and imaginary parts and reconstructs an image therefrom according to known techniques. System 20 may comprise any general-purpose or dedicated computing system. Accordingly, system 20 includes one or more processing units 21 (e.g., processors, processor cores, execution threads, etc.) configured to execute processor-executable program code to cause system 20 to operate as described herein, and storage device 22 for storing the program code. Storage device 22 may comprise one or more fixed disks, solid-state random access memory, and/or removable media (e.g., a thumb drive) mounted in a corresponding interface (e.g., a USB port).

Storage device 22 stores program code of control program 23. One or more processing units 21 may execute control program 23 to cause system 20 to perform any one or more of the processes described herein. For example, one or more processing units 21 may execute control program 23 to cause system 20 to execute a pulse sequence according to a displacement-encoding imaging technique. Pulse sequences 26 include data specifying the parameters of such pulse sequences and their constituent building blocks and readout events.

One or more processing units 21 may execute control program 23 to cause system 20 to receive the real and imaginary parts of a received RF signal via MR system interface 24 and reconstruct an image therefrom according to known techniques. Such an image may be stored among acquired images 27 of storage device 22. Reconstruction may also include correction for background phase offset error as described herein and/or any other image correction process that is or becomes known. Thusly-corrected images may be stored among corrected images 28. Displacement maps 29 may include displacement information determined based on phase offset error-corrected displacement-encoded phase images as described herein.

One or more processing units 21 may also execute control program 23 to provide instructions to sequence controller 10 via MR system interface 24. For example, sequence controller 10 may be instructed to initiate a desired pulse sequence of pulse sequences 26. In particular, sequence controller 10 may be instructed to control the switching of magnetic field gradients via amplifiers 8a-8c at appropriate times, the transmission of radio-frequency pulses having a specified phase and amplitude at specified times via RF system 11 and RF amplifier 12, and the readout of the resulting magnetic resonance signals.

Acquired images 27, corrected images 28 and/or displacement maps 29 may be provided to terminal 30 via terminal interface 25 of system 20. Terminal interface 25 may also receive input from terminal 30, which may be used to provide commands to control program 23 in order to control sequence controller 10 and/or other elements of system 1. Terminal 30 may simply comprise a display device and an input device coupled to system 20. In some embodiments, terminal 30 is a separate computing device such as, but not limited to, a desktop computer, a laptop computer, a tablet computer, and a smartphone.

Each element of system 1 may include other elements which are necessary for the operation thereof, as well as additional elements for providing functions other than those described herein. Storage device 22 may also store data and other program code for providing additional functionality and/or which are necessary for operation of system 20, such as device drivers, operating system files, etc.

The foregoing diagrams represent logical architectures for describing processes according to some embodiments, and actual implementations may include more or different components arranged in other manners. Other topologies may be used in conjunction with other embodiments. Moreover, each component or device described herein may be implemented by any number of devices in communication via any number of other public and/or private networks. Two or more of such computing devices may be located remote from one another and may communicate with one another via any known manner of network(s) and/or a dedicated connection. Each component or device may comprise any number of hardware and/or software elements suitable to provide the functions described herein as well as any other functions. For example, any computing device used in an implementation of a system according to some embodiments may include a processor to execute program code such that the computing device operates as described herein.

All systems and processes discussed herein may be embodied in program code stored on one or more non-transitory computer-readable media. Such media may include, for example, a floppy disk, a CD-ROM, a DVD-ROM, a Flash drive, magnetic tape, and solid state Random Access Memory (RAM) or Read Only Memory (ROM) storage units. Embodiments are therefore not limited to any specific combination of hardware and software.

Embodiments described herein are solely for the purpose of illustration. Those in the art will recognize other embodiments may be practiced with modifications and alterations to that described above.

What is claimed is:

1. A system comprising:
a chassis defining a bore;
a main magnet to generate a polarizing magnetic field within the bore;
a gradient system to apply a gradient magnetic field to the polarizing magnetic field;
a radio frequency system to transmit RF pulses to patient tissue disposed within the bore and to receive signals from the patient tissue; and
a computing system to execute program code to:
control the gradient system and radio frequency system to acquire displacement-encoded k-space data;
generate a plurality of displacement-encoded magnetic resonance (MR) phase images based on the k-space data;
determine first pixels associated with one or more substantially stationary image regions of the plurality of displacement-encoded MR phase images;
determine representative pixel values of the first pixels based on pixel values of the first pixels within one or more of the plurality of displacement-encoded MR phase images;
determine a relationship between background phase offset error and pixel location based on the determined representative pixel values and the pixel locations of the first pixels;
determine a background phase offset error for each of one or more other pixels of the plurality of displacement-encoded MR phase images based on the relationship and on the pixel locations of the one or more other pixels; and
generate a corrected MR phase image of one of the plurality of displacement-encoded MR phase images based on the background phase offset error determined for each of the one or more other pixels.

2. A system according to claim 1, the computing system to execute program code to:
generate a corrected MR phase image of a second one of the plurality of displacement-encoded MR phase images based on the background phase offset error determined for each of the one or more other pixels; and
determine a displacement map based on the corrected MR phase image of the one of the plurality of displacement-encoded MR phase images and the corrected MR phase image of the second one of the plurality of displacement-encoded MR phase images.

3. A system according to claim 1, wherein determination of the first pixels associated with the one or more image regions comprises:
determination that a standard deviation of pixel values of the first pixels within the plurality of displacement-encoded MR phase images is below a threshold value.

4. A system according to claim 1, the computing system to execute program code to:
determine second pixels associated with noisy image regions of the plurality of displacement-encoded MR phase images, and
wherein the first pixels do not include any of the second pixels.

5. A system according to claim 4, wherein determination of the second pixels comprises determination of the second pixels based on one or more MR magnitude images corresponding to one or more of the plurality of displacement-encoded MR phase images.

6. A system according to claim 1, further comprising:
a display terminal to display the displacement map.

7. A system according to claim 1, wherein generation of the corrected MR phase image comprises subtraction of the background phase offset error determined for each of the one or more other pixels from the pixel values of the one or more other pixels of the one of the plurality of displacement-encoded MR phase images.

8. A computer-implemented method comprising:
acquiring a plurality of displacement-encoded magnetic resonance (MR) phase images;
determining first pixels associated with one or more image regions of the plurality of displacement-encoded MR phase images;
determining representative pixel values of the first pixels based on pixel values of the first pixels within one or more of the plurality of displacement-encoded MR phase images;
determining a relationship between background phase offset error and pixel location based on the determined representative pixel values and the pixel locations of the first pixels;
determining a background phase offset error for each of one or more other pixels of the plurality of displacement-encoded MR phase images based on the relationship and on the pixel locations of the one or more other pixels; and
generating a corrected MR phase image of one of the plurality of displacement-encoded MR phase images based on the background phase offset error determined for each of the one or more other pixels.

9. A method according to claim 8, further comprising:
generating a corrected MR phase image of a second one of the plurality of displacement-encoded MR phase images based on the background phase offset error determined for each of the one or more other pixels; and
determining a displacement map based on the corrected MR phase image of the one of the plurality of displacement-encoded MR phase images and the corrected MR phase image of the second one of the plurality of displacement-encoded MR phase images.

10. A method according to claim 8, wherein determining the first pixels associated with the one or more image regions comprises:
determining that the first pixels are associated with substantially stationary image regions.

11. A method according to claim 10, wherein determining that the first pixels are associated with substantially stationary image regions comprises:
determining that a standard deviation of pixel values of the first pixels within the plurality of displacement-encoded MR phase images is below a threshold value.

12. A method according to claim 10, further comprising:
determining second pixels associated with noisy image regions of the plurality of displacement-encoded MR phase images, and
wherein the first pixels do not include any of the second pixels.

13. A method according to claim 12, wherein determining the second pixels comprises determining the second pixels based on one or more MR magnitude images corresponding to one or more of the plurality of MR phase images.

14. A system comprising:
a computing system to:
acquire a plurality of displacement-encoded magnetic resonance (MR) phase images;
determine first pixels associated with one or more image regions of the plurality of displacement-encoded MR phase images;
determine representative pixel values of the first pixels based on pixel values of the first pixels within one or more of the plurality of displacement-encoded MR phase images;
determine a relationship between background phase offset error and pixel location based on the determined representative pixel values and the pixel locations of the first pixels;
determine a background phase offset error for each of one or more other pixels of the plurality of displacement-encoded MR phase images based on the relationship and on the pixel locations of the one or more other pixels;
generate a corrected MR phase image of one of the plurality of displacement-encoded MR phase images based on the background phase offset error determined for each of the one or more other pixels; and
display the corrected MR phase image.

15. A system according to claim 14, the computing system to:
generate a corrected MR phase image of a second one of the plurality of displacement-encoded MR phase images based on the background phase offset error determined for each of the one or more other pixels;
determine a displacement map based on the corrected MR phase image of the one of the plurality of displacement-encoded MR phase images and the corrected MR phase image of the second one of the plurality of displacement-encoded MR phase images; and
display the displacement map.

16. A system according to claim 14, wherein determination of the first pixels associated with the one or more image regions comprises:
determination that the first pixels are associated with substantially stationary image regions.

17. A system according to claim 16, wherein determination that the first pixels are associated with substantially stationary image regions comprises:
determination that a standard deviation of pixel values of the first pixels within the plurality of displacement-encoded MR phase images is below a threshold value.

18. A system according to claim 14, the computing system to:
determine second pixels associated with noisy image regions of the plurality of displacement-encoded MR phase images, and
wherein the first pixels do not include any of the second pixels.

19. A system according to claim 18, wherein determination of the second pixels comprises determination of the second pixels based on one or more MR magnitude images corresponding to one or more of the plurality of displacement-encoded MR phase images.
